(19) United States
(12) Patent Application Publication
(10) Pub. No.: US 2017/0000853 A1
(43) Pub. Date: Jan. 5, 2017

(54) WOUND HEALING COMPOSITION

(71) Applicant: Guido Wouters, Putte (BE)

(72) Inventor: Guido Wouters, Putte (BE)

(21) Appl. No.: 15/196,116

(22) Filed: Jun. 29, 2016

(30) Foreign Application Priority Data
Jun. 30, 2015 (BE) 2015/5411

(51) Int. Cl. A61K 38/36; A61K 33/06; A61K 31/4045; A61K 31/417; A61Q 7/00; A61K 38/20; A61K 38/43; A61K 8/64; A61K 8/66; A61K 8/49; A61K 38/18; A61K 38/19

(52) U.S. Cl. CPC A61K 38/363; A61K 38/1841; A61K 38/1858; A61K 38/1808; A61K 38/1866; A61K 38/36; A61K 33/06; A61K 31/4045; A61K 31/417; A61K 38/18; A61K 38/185; A61K 38/193; A61K 38/204; A61K 38/2046; A61K 38/2053; A61K 38/2073; A61K 38/208; A61K 38/2093; A61K 38/191; A61K 38/43; A61K 8/64; A61K 8/66; A61K 8/4946; A61K 8/492; A61Q 7/00

(57) ABSTRACT

The present invention relates to compositions for use in pharmaceutical, cosmetic and cosmeceutical applications, particularly wound healing, including the treatment of lesions and burns. In particular, the present invention relates to compositions comprising platelet enriched plasma (PRP) and stem cell conditioned medium (SC CM) for use in pharmaceutical, cosmetic and cosmeceutical applications such as wound healing.

### CROSS-REFERENCE TO RELATED APPLICATION

This application claims priority to Belgian Patent Application No. 2015/5411, filed Jun. 30, 2015, the contents of which are herein incorporated by reference in their entirety.

### FIELD OF THE INVENTION

The present invention relates to compositions for use in pharmaceutical, cosmetic and cosmeceutical applications, particularly wound healing, including the treatment of lesions and burns. In particular, the present invention relates to compositions comprising platelet enriched plasma (PRP) and stem cell conditioned medium (SC CM) for use in pharmaceutical, cosmetic and cosmeceutical applications such as wound healing.

### BACKGROUND OF THE INVENTION

Wound healing is a process which passes through several phases. Initially, in the inflammatory phase the body responds to the injury by contracting the blood vessels in the wound bed and the formation of a clot. Once haemostasis has been achieved, blood vessels then dilate to allow essential cells; antibodies, white blood cells, growth factors, enzymes and nutrients to reach the wounded area. This leads to a rise in exudate levels so the surrounding skin needs to be monitored for signs of maceration.

It is at this stage that the characteristic signs of inflammation can be seen; erythema, heat, oedema, pain and functional disturbance. The predominant cells at work here are the phagocytic cells; ‘neutrophils and macrophages’; mounting a host response and autolysing any devitalised ‘necrotic/sloughy’ tissue.

During proliferation, the wound is ‘rebuilt’ with new granulation tissue which is comprised of collagen and extracellular matrix and into which a new network of blood vessels develop, a process known as ‘angiogenesis’. Healthy granulation tissue is dependent upon the fibroblast receiving sufficient levels of oxygen and nutrients supplied by the blood vessels. Healthy granulation tissue is granular and uneven in texture; it does not bleed easily and is pink/red in colour. The colour and condition of the granulation tissue is often an indicator of how the wound is healing. Dark granulation tissue can be indicative of poor perfusion, ischaemia and/or infection. Epithelial cells finally resurface the wound, a process known as ‘epithelialisation’.

Maturation is the final phase and occurs once the wound has closed. This phase involves remodeling of collagen from type III to type I. Cellular activity reduces and the number of blood vessels in the wounded area regress and decrease.

Stem cells are of great interest in numerous therapeutic, cosmetic and cosmeceutical areas because of their capacity to form cells of multiple types. The culture media used to grow cells, including stem cells, have been described for therapeutic, cosmetic and cosmeceutical uses arising from the secretion by the growing cells of proteins and other factors into the media. See for example U.S. Pat. No. 7,118.746; U.S. Pat. No. 7,160,726 and WO2008/020815.

Also, platelet enriched plasma (PRP), a product of blood plasma that is rich in platelets, is known for use in a variety of therapeutic or cosmetic applications including enhancing wound healing in dental implants and sinus elevations, heart surgery, orthopaedic surgery and dermatology (chronic wound healing). For instance WO2005/065269 discloses several compositions comprising PRP and fibroblast cells for the treatment of skin, in particular, repeated administration of PRP in a dermatologically acceptable carrier to skin to e.g. reduce appearance of wrinkles.

Despite the above, there remains a need for alternative compositions for therapeutic, cosmetic and cosmeceutical purposes. It is particularly desirable to identify new types of compositions which work more efficiently compared to the already existing products.

### SUMMARY OF THE INVENTION

In a first aspect, the present invention relates to a composition comprising platelet enriched plasma (PRP) and stem cell conditioned medium (SC CM).

More particular, in the composition according to the present invention said PRP comprises transforming growth factor-β (TGF-β), fibrinogen, platelet-derived growth factor (PDGF), epidermal growth factor (EGF), transforming growth factor-α (TGF-α), vascular endothelial growth factor (VEGF), platelet thrombo-plastin, thrombospondin, coagulation factors, calcium, serotonin, histamine, and hydrolytic enzymes.

More particular, in the composition according to the present invention said SC CM is the medium harvested after the culturing of mesenchymal stem cells.

More particular, in the composition according to the present invention said SC CM comprises hepatocyte growth factor, transforming growth factor β (TGF-β), anti-apoptopic factors, keratinocyt growth factor, brain-derived neurotrophic factor (BDNF), Flt-3 ligand, granulocyte colony stimulating factor (G-CSF), granulocyte/macrophage colony stimulating factor (GM-CSF), macrophage colony stimulating factor (M-CSF), interleukin-6 (IL-6), interleukin-7 (IL-7), interleukin-8 (IL-8), interleukin-11 (IL-11), interleukin leukin-12 (IL-12), leukemia inhibitory factor (LIF), and tumor necrosis factor-alpha (TNF-α).

In particular, the composition according to the present invention further comprises DMSO.

In particular, the composition according to the present invention further comprises a gelling agent.

More particular, in the composition according to the present invention said PRP and SC CM are present in the composition in a ratio of about 3:1 to about 1:3.

In particular, in the composition according to the present invention said SC CM is prepared from stem cells isolated from bone marrow or adipose tissue. Particularly, said stem cells are mesenchymal stem cells.

More particular, in the composition according to the present invention said conditioned medium is a processed conditioned medium.

According to a second aspect, the present invention relates to the compositions as described herein for use in (animal) medicine, more in particular for use in the treatment of tissue injuries in a subject and/or mastitis. Preferably said tissue injuries comprise skin burns, tendon injuries, ulcers, skin wounds, and wherein said tissue injuries are preferably skin wounds. Preferably, said subject is a mammal, preferably selected from domestic animals such as dogs, cats and rabbits or farm animals such as horses, cattle, sheep and goats.

According to a third aspect, the present invention relates to the use of the compositions as described herein for promoting hair growth in a mammalian subject.

According to a third aspect, the present invention relates to a method for preparing a composition according to the present invention, comprising at least the blending of platelet enriched plasma (PRP) and stem cell conditioned medium (SC CM).

### BRIEF DESCRIPTION OF FIGURES

FIG. 1A-1B shows the effect of the composition of the invention on wounds in hedgehogs.

FIG. 2A-2B shows the effect of the composition of the invention on wounds in hedgehogs.

FIG. 3A-3B shows the effect of the composition of the invention on wounds in horses.

FIG. 4A-4B shows the effect of the composition of the invention on wounds in dogs.

FIG. 5A-5B shows the effect of the composition of the invention on wounds in mice.

FIG. 6A-6B shows the effect of the composition of the invention on wounds in elephants.

FIG. 7 is a representation of the wound closure. Wound area is calculated from the average of three daily diameter measurements along the x, y and z-axes. Wound closure is expressed as a percentage of initial wound area at day 0.

### DETAILED DESCRIPTION OF THE INVENTION

Before the present method and devices used in the invention are described, it is to be understood that this invention is not limited to particular methods, components, or devices described, as such methods, components, and devices may, of course, vary. It is also to be understood that the terminology used herein is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein may be used in the practice or testing of the present invention, the preferred methods and materials are now described.

In this specification and the appended claims, the singular forms “a”, “an”, and “the” include plural references unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The terms “comprising”, “comprises” and “comprised of” also include the term “consisting of”.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The recitation of numerical ranges by endpoints includes all numbers and fractions subsumed within the respective ranges, as well as the recited endpoints.

The present invention provides in compositions comprising both platelet enriched plasma (PRP) and stem cell conditioned medium (SC CM).

The term “PRP” or “platelet rich plasma” as used herein should be understood to mean a blood product which comprises platelets concentrate in a small volume of plasma. Platelet enriched plasma (PRP) is obtained by processing blood to obtain platelet enriched plasma for instance by double centrifugation designed to separate the PRP aliquot from platelet-poor plasma and red blood cells.

The term “SC CM” or “stem cell conditioned medium” as used herein should be understood to mean a medium that has been harvested from cultured cells, typically cultured stem cells. Stem cell conditioned medium typically contains metabolites, growth factors, and extracellular matrix proteins secreted into the medium by the cultured stem cells. Examples of each might include: metabolites such as glucose, amino acids, and nucleosides; growth factors such as interleukins, EGF (epidermal growth factor), and PDGF (platelet-derived growth factor); and matrix proteins such as collagen, fibronectin, and various proteoglycans.

While therapeutic treatments with platelet rich plasma and stem cell conditioned medium separately have been shown great promises, it has now been found that the combination of both creates a synergistic effect. In particular for the healing of wounds, typical healing times with the products on the market range between 3 to 6 weeks, while the combination of platelet enriched plasma and stem cell conditioned medium allows a further reduction of the healing time to 1 to 2 weeks.

It is important in the healing of surgical or traumatic wounds that the healing occurs quick and without delays, in the interest of the costs as well as the success of the healing process. The composition according to the invention has been found to provide in a successful and fast cutaneous wound healing process. A rich source of the complex group of growth factors in the product allows the natural repair of the wound. The platelets act in the haemostasis; wound healing and re-epithelialization liberating diverse GF's that stimulate the angiogenesis, promoting growth and vascular fibroblast proliferation that in turn provide an increase in the collagen synthesis. Both Platelet-rich plasma (PRP) and stem cell conditioned medium (SC CM) is 100% biocompatible and safe. The healing rate and the regaining of the original skin reveals the high regenerative potential.

Accordingly, in a first aspect, the present invention therefore provides in compositions comprising platelet enriched plasma (PRP) and stem cell conditioned medium (SC CM).

Preferably, in the compositions according to the invention said PRP comprises at least transforming growth factor-β (TGF-β), fibrinogen, platelet-derived growth factor (PDGF), epidermal growth factor (EGF), transforming growth factor-α (TGF-α), vascular endothelial growth factor (VEGF), platelet thrombo-plastin, thrombospondin, coagulation factors, calcium, serotonin, histamine, and hydrolytic enzymes.

To the PRP fraction also other substances may be added including for instance anti-oxidants such as vitamins such as vitamin C (ascorbic acid), vitamin E, vitamin A and other retinoids; and the carotenes such as beta.-carotene.

Preferably, in the compositions according to the invention said SC CM is the medium harvested after the culturing of mesenchymal stem cells.

A particular synergetic effect was observed when the cells from which the conditioned medium is harvested were mesenchymal stem cells. Mesenchymal stem cells (MSCs) are multipotent stromal cells that can differentiate into a variety of cell types, including: osteoblasts (bone cells), chondrocytes (cartilage cells), myocytes (muscle cells) and adipocytes (fat cells). Typically, MSCs are harvested from bone marrow, adipose tissue, the placenta, the umbilical cord blood, adult muscle, corneal stroma or the dental pulp of deciduous baby teeth and cultured for a certain period in time (typically 2 weeks) before harvesting the cells and the conditioned medium. The conditioned medium is rich in growth factors such as hepatocyte growth factor, transforming growth factor β, anti-apoptopic factors, keratinocyt growth factor, brain-derived neurotrophic factor (BDNF), Flt-3 ligand, granulocyte colony stimulating factor (G-CSF), granulocyte/macrophage colony stimulating factor (GM-CSF), macrophage colony stimulating factor (M-CSF), interleukin-6 (IL-6), interleukin-7 (IL-7), interleukin-8 (IL-8), interleukin-11 (IL-11), interleukin leukin-12 (IL-12), leukemia inhibitory factor (LIF), and tumor necrosis factor-alpha (TNF-α).

Accordingly, in the composition according to the present invention said SC CM comprises at least hepatocyte growth factor, transforming growth factor β (TGF-β), anti-apoptopic factors, keratinocyt growth factor, brain-derived neurotrophic factor (BDNF), Flt-3 ligand, granulocyte colony stimulating factor (G-CSF), granulocyte/macrophage colony stimulating factor (GM-CSF), macrophage colony stimulating factor (M-CSF), interleukin-6 (IL-6), interleukin-7 (IL-7), interleukin-8 (IL-8), interleukin-11 (IL-11), interleukin leukin-12 (IL-12), leukemia inhibitory factor (LIF), and tumor necrosis factor-alpha (TNF-α).

To the SC CM fraction also other substances may be added including for instance Ca++ ions which can activate the platelets of the PRP substances.

Preferably, in the composition according to the present invention the stem cells from which the SC CM is obtained are isolated from bone marrow or adipose tissue.

In particular, the composition according to the present invention further comprises DMSO. Dimethyl sulfoxide (DMSO) is an organosulfuric compound with the formula (CH₃)₂SO. DMSO is a colourless liquid and an important polar aprotic solvent that dissolves both polar and nonpolar compounds, miscible in a wide range of organic solvents as well as water. The addition of DMSO to the composition according to the invention was found to be particularly helpful and additionally improved the effectivity of the composition for the treatment of tissue injuries in a subject, and in particular for wound healing purposes.

By using the composition comprising PRP and SC CM combined with DMSO a very efficient wound healing process has been observed. The wound closure time is much shortened even further and the healing is better (granulation, pink colour, closure time). The different phases inflammation phase, proliferation phase and maturation phase are shortened.

Preferably DMSO is present in the composition according to the present invention in amount ranging between 5% and 25%, preferably between 7.5% and 20% and more preferably about 10%, 15% or 20%.

Also, while the composition according to the present invention may be used in liquid form (in a treatment bath or as a spray), the effectivity may be further improved by providing the composition according to the invention in the form of a gel. Accordingly, the composition according to the invention preferably further comprises a gelling agent. Accordingly, the composition according to the invention can be applied as a topical treatment.

In preferred embodiments, the ratio in which said PRP and SC CM are present in the composition according to the invention range between about 3:1 to about 1:3, preferably between about 2:1 to about 1:2, and more preferably 2:1, 1:1 or 1:2.

In a particular embodiment, in the composition according to the present invention conditioned medium is a processed conditioned medium. The term “processed conditioned medium” refers to a conditioned medium that has undergone several treatments such as washing, purification and/or feeding steps. The medium preferably harvested after a confluent growth of the cell culture.

In a second aspect, the present invention relates to a composition according to the above for use in medicine, preferably animal medicine.

In particular the composition according to the invention has been shown to have an increased and synergetic clinical effect in the regeneration of the tissue (wound healing). It has been found that the composition promotes tissue epithelialisation and granulation of wounds. It can be applied on superficial and deep wounds as a result of a trauma, surgery or any other external tissue damage where skin closure is a problem. The compositions according to the invention have been found to heal wounds more rapidly compared to conventional treatments and the compositions according to the invention can therefore be used to accelerate the healing process and reduce scar formation.

Preferably, the present invention relates to a composition according to the above for use in the treatment of tissue injuries in a subject.

It has also been found that both the PRP and the SC CM as present in the compositions according to the invention may be from either “autogenic” donors or “allogeneic” donors. This simplifies the treatment as it is no longer necessary that the donor of the source of the compositions according to the invention, the donor of the stem cells or the blood, is to be the same as the receiver of the treatment. Autogenic methods which typically use the patient's own blood and stem cells are more expensive as they require an individualized approach, while for an allogeneic method blood and/or stem cells donated by one or more third parties can be used for preparing the compositions. This allows the production of larger quantities, hence reducing the production costs drastically.

Accordingly, the present invention relates to a composition according to the above for use in the allogeneic treatment of tissue injuries in a subject.

Preferably, said tissue injuries comprise skin burns, tendon injuries, ulcers, skin wounds, and wherein said tissue injuries are preferably skin wounds.

Preferably said subject is a mammal, preferably selected from domestic animals such as dogs, cats and rabbits or farm animals such as horses, cattle, sheep and goats.

The compositions according to the invention are applied on a cleaned wound. The composition, preferably in the form of a gel, can be applied on the wound directly or using a sterile dressing. Typically only a single application on the wound is sufficient, but depending on the size and the appearance of the wound treatment can be repeated in a week interval.

In a further aspect, the present invention relates to a composition according to the above for use in the treatment of mastitis in a subject.

The compositions according to the invention has been shown to reduce the inflammation of the mammary glands and to promote the healing of wounds which can occur in the nipple tissue during mastitis.

As described above, it has also been found that both the PRP and the SC CM as present in the compositions according to the invention may be from either “autogenic” donors or “allogeneic” donors.

Accordingly, the present invention relates to a composition according to the above for use in the allogeneic treatment of tissue injuries in a subject.

In a further aspect, the present invention relates to the use of a composition according to the above, for promoting hair growth as an essential part of skin regeneration in a mammalian subject.

Additionally, it has been observed that the compositions according to the invention and as described above are shown to promote hair growth in mammals. Preferably, the present invention relates to the use of the compositions according to the invention to promote hair growth in mammals wherein the hair is selected from those on the top of the head, on the armpits, on the pubic area, on the face including eyelash, eyebrow, eyelid, moustache, beard and whisker, on the chest, arms and legs, preferably hair is selected from those on scalp, beard, head, pubic area, upper lip, eyelash, eyebrow, and eyelid.

In a further aspect, the present invention relates to a method for preparing a composition according to the invention, comprising at least the blending of platelet enriched plasma (PRP) and stem cell conditioned medium (SC CM). The process includes the collection of the secretes of mesenchymal stem cells that activates the gelling of the platelet enriched plasma. The formation of a glue appears after about 6 hours.

The present invention is hereafter exemplified by the illustration of particular, non-limiting examples.

### EXAMPLES

### Example 1

### Wound Healing Study

A study was conducted using the composition according to the invention. The composition consisted of 30% of platelet enriched plasma, 60% of Mesenchymal Stem Cell Conditioned Medium and 10% of DMSO.

Hedgehogs which were diagnosed with skin wounds were treated with the treatment composition comprising platelet enriched plasma and Mesenchymal Stem Cell Conditioned medium. An overview of the treatment outcome is given in table 1.

TABLE 1

| | | Treatment (days) | | | | | |
|---|---|---|---|---|---|---|---|
| Subject | Wound description | 0 | 2 | 5 | 7 | 8 | Result |
| 1 | Small wound on | A | + | +++ | − | RA | Healed |
| | right elbow | | | | | | |
| 2 | Long lasting | A | + | +++ | +++ | RA | Healed |
| | wound on face | | | | | | |
| 3 | Infection of | A | + | +++ | +++ | RA | Healed |
| | the eye orbit | | | | | | |
| 4 | Skin infection | A | / | +++ | + | | Healed |
| | and sarcoptes | | | | | | |
| 5 | Deep wound on jaw | A | − | − | + | | Improved |

A = Application of the treatment composition
RA = Reapplication of the treatment composition
− = No further improvement
+ = Minor improvement
+++ = Great improvement

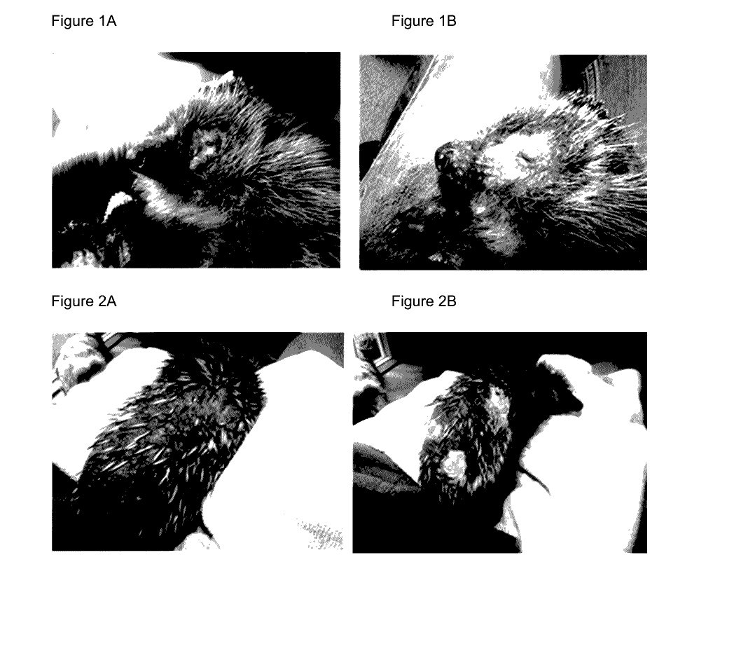

FIGS. 1A and B show the improvement over time of the wound of subject 3

FIGS. 2A and B show the improvement over time of the wound of subject 4

### Example 2

### Hair Growth Study

Hedgehogs which were at least partially bold because of scabies were treated with the treatment composition comprising platelet enriched plasma and Mesenchymal Stem Cell Conditioned medium. The boldness was caused by damage to the roots of the spines.

The composition as used in example 1 was applied and after 10 days it was observed that new spines appeared.

### Example 3

### Wound Healing Study

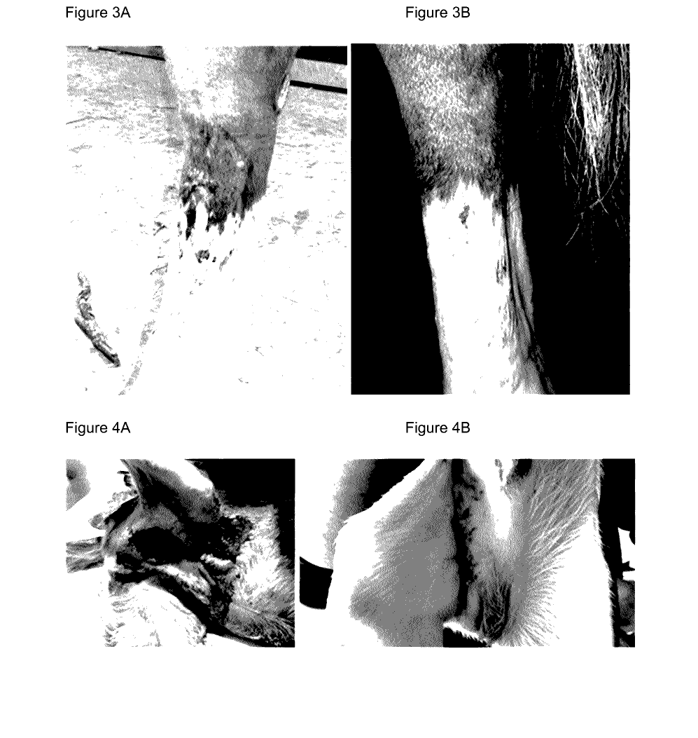

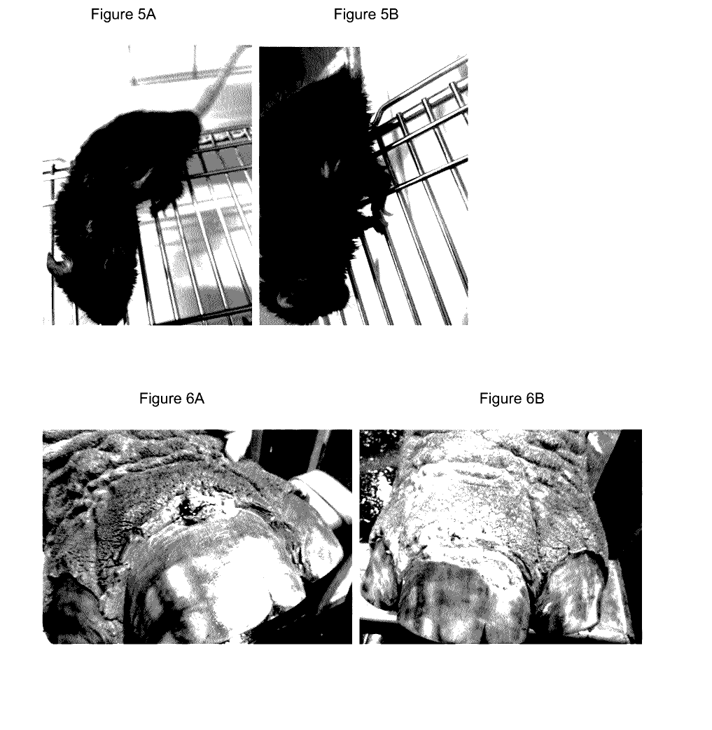

The composition as used in example 1 was also successfully used to treat wounds in horses, dogs, mice suffering from ulcerative dermatitis and elephants affected by hoof lesions. The composition as used in example 1 was applied to severe wounds in horses (FIG. 3A), dogs (FIG. 4A), mice (FIG. 5A) and elephants (FIG. 6A) and after about two weeks the wounds were almost completely healed (FIGS. 3B, 4B, 5B and 6B).

### Example 4

### Wound Healing Study

Experimental Animals

All animal experiments were approved by the Animal Ethics Committee. Sixty 8-week old (22-26 grams) male/female mice were kept in standard conditions of 21° C. and a normal light-dark cycle with free access to food and water.

Anesthesia and Operative Preparation

General anesthesia was induced using 5% isoflurane in 100% oxygen (flow rate 1 L/min) and maintained using 1-3% isoflurane. The suppression of deep pedal reflexes of the mouse was ensured and the mouse was placed in the prone position. Next, the operative region was prepared by removing to 3cm round the place of cutting between the two shoulder blades of the mouse. Subsequently, the skin was wiped with an alcohol swab and two applications of 10% povidone-iodine (Betadine) and draped.

Excision

A scalpel was used to make a 1 cm longitudinal injury on either side of the mouse's midline at the level of the shoulders. Next, a serrated forceps was used to lift the skin in the middle of the outline and iris scissors to create a full-thickness wound that extends through the subcutaneous tissue. This process was repeated for the wound on the other side of the midline. The therapeutic compound was applied to one wound (right side) and the control to the other (left side) (see Table 2). Following topical applications were applied: growth factors from blood (product A), growth factors from adipose stem cells (product B), or both (product A+B in gel) (see Table 2). Finally, the wound was not sutured and no special second dressing was used.

Postoperative Management

Carprofen (5 mg/kg) was administered once daily via sub-cutaneous injection for post-operative pain relief. After the surgery animals were caged individually and maintained until fully recovered. The animals were monitored twice daily for manifestations of pain and weight loss. No gross behavioral displays of pain or weight loss were observed.

TABLE 2

| | 20 mice with U.D. | | 20 mice with U.D. | |
|---|---|---|---|---|
| Group U.D. | Treated with AB gel | | Not treated | |
| Side of wound | Left | Right | Left | Right |
| Group 1 (=10 + 10 mice) | Vehicle | A | A | B |
| Group 2 (=10 + 10 mice) | Vehicle | B | B | A |
| Group 3 (=10 + 10 mice) | A | AB | B | AB |

Wound Measurement and Treatment

The wound was measured daily. General anesthesia was induced using 5% isoflurane gas (flow rate 1 L/min), and subsequently the suppression of deep sensory reflexes of the mouse was ensured using 1-3% isoflurane. Next, the occlusive dressing was peeled back gently with forceps. Surgical calipers were used to measure the wound diameter. The average of three measurements was taken along the X, Y and Z. When considered necessary, the therapeutic compound and the vehicle control were re-applied at this point. Finally, a clean transparent occlusive dressing was re-applied and the animals were kept warm until fully recovered. At the completion of experiments, wound closure, morphological architecture and degree of neovascularization, granulation were observed and scored.

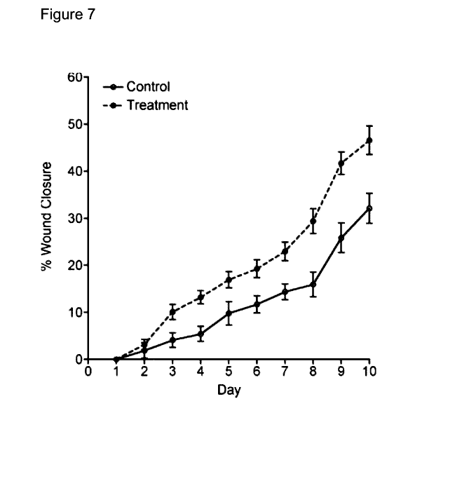

A wound closure curve was determined by calculating the average diameter of the wound and expressing the results as a percentage, i.e. 100−(Day 0 diameter/Day×diameter). In this experiment a therapeutic compound (or vehicle control) was applied daily to the wound. The therapeutic compound greatly accelerated wound closure (FIG. 7).

1. A composition comprising platelet enriched plasma (PRP) and stem cell conditioned medium (SC CM).

2. The composition according to claim 1, wherein said PRP comprises transforming growth factor-β (TGF-β), fibrinogen, platelet-derived growth factor (PDGF), epidermal growth factor (EGF), transforming growth factor-α (TGF-α), vascular endothelial growth factor (VEGF), platelet thrombo-plastin, thrombospondin, coagulation factors, calcium, serotonin, histamine, and hydrolytic enzymes.

3. The composition according to claim 1, wherein said SC CM is the medium harvested after the culturing of mesenchymal stem cells.

4. The composition according to claim 2, wherein said SC CM is the medium harvested after the culturing of mesenchymal stem cells.

5. The composition according to claim 1, wherein said SC CM comprises hepatocyte growth factor, transforming growth factor β (TGF-β), anti-apoptopic factors, keratinocyt growth factor, brain-derived neurotrophic factor (BDNF), Flt-3 ligand, granulocyte colony stimulating factor (G-CSF), granulocyte/macrophage colony stimulating factor (GM-CSF), macrophage colony stimulating factor (M-CSF), interleukin-6 (IL-6), interleukin-7 (IL-7), interleukin-8 (IL-8), interleukin-11 (IL-11), interleukin leukin-12 (IL-12), leukemia inhibitory factor (LIF), and tumor necrosis factor-alpha (TNF-α).

6. The composition according to claim 2, wherein said SC CM comprises hepatocyte growth factor, transforming growth factor β (TGF-β), anti-apoptopic factors, keratinocyt growth factor, brain-derived neurotrophic factor (BDNF), Flt-3 ligand, granulocyte colony stimulating factor (G-CSF), granulocyte/macrophage colony stimulating factor (GM-CSF), macrophage colony stimulating factor (M-CSF), interleukin-6 (IL-6), interleukin-7 (IL-7), interleukin-8 (IL-8), interleukin-11 (IL-11), interleukin leukin-12 (IL-12), leukemia inhibitory factor (LIF), and tumor necrosis factor-alpha (TNF-α).

7. The composition according to claim 3, wherein said SC CM comprises hepatocyte growth factor, transforming growth factor β (TGF-β), anti-apoptopic factors, keratinocyt growth factor, brain-derived neurotrophic factor (BDNF), Flt-3 ligand, granulocyte colony stimulating factor (G-CSF), granulocyte/macrophage colony stimulating factor (GM-CSF), macrophage colony stimulating factor (M-CSF), interleukin-6 (IL-6), interleukin-7 (IL-7), interleukin-8 (IL-8), interleukin-11 (IL-11), interleukin leukin-12 (IL-12), leukemia inhibitory factor (LIF), and tumor necrosis factor-alpha (TNF-α).

8. The composition according to claim 4, wherein said SC CM comprises hepatocyte growth factor, transforming growth factor β (TGF-11), anti-apoptopic factors, keratinocyt growth factor, brain-derived neurotrophic factor (BDNF), Flt-3 ligand, granulocyte colony stimulating factor (G-CSF), granulocyte/macrophage colony stimulating factor (GM-CSF), macrophage colony stimulating factor (M-CSF), interleukin-6 (IL-6), interleukin-7 (IL-7), interleukin-8 (IL-8), interleukin-11 (IL-11), interleukin leukin-12 (IL-12), leukemia inhibitory factor (LIF), and tumor necrosis factor-alpha (TNF-α).

9. The composition according to claim 1, wherein said composition further comprises DMSO.

10. The composition according to claim 1, wherein said composition further comprises a gelling agent.

11. The composition according to claim 1, wherein said PRP and SC CM are present in the composition in a ratio of about 3:1 to about 1:3.

12. The composition according to claim 1, wherein the stem cells are isolated from bone marrow or adipose tissue.

13. The composition according to claim 1, wherein the stem cells are mesenchymal stem cells.

14. The composition according to claim 1, wherein said conditioned medium is a processed conditioned medium.

15. A method of treating tissue injuries in a subject using the composition according to claim 1.

16. A method of treating tissue injuries in a subject using the composition according to claim 1 wherein said tissue injuries comprise skin burns, tendon injuries, ulcers, skin wounds, and wherein said tissue injuries are preferably skin wounds.

17. A method of treating tissue injuries in a subject using the composition according to claim 1, wherein said subject is a mammal, preferably selected from domestic animals such as dogs, cats and rabbits or farm animals such as horses, cattle, sheep and goats.

18. A method of promoting hair growth in a mammalian subject using a composition according to claim 1.

19. A method for preparing a composition according to claim 1, comprising at least the blending of platelet enriched plasma (PRP) and stem cell conditioned medium (SC CM).
